(19) United States
(12) Patent Application Publication
(10) Pub. No.: US 2025/0295307 A1
(43) Pub. Date: Sep. 25, 2025

(54) A DEVICE AND A METHOD FOR AUTOMATED MEASUREMENTS OF EYEBALL DEVIATION AND/OR VERTICAL AND HORIZONTAL VIEWING ANGLES

(71) Applicant: CX ENGINEERING SPOLKA Z OGRANICZONA ODPOWIEDZIALNOSCIA, Balice (PL)

(72) Inventors: Monika MODRZEJEWSKA, Przeclaw (PL); Magdalena DURAJCZYK, Mierzyn (PL); Ewa GRUDZINSKA, Mierzyn (PL)

(21) Appl. No.: 18/863,324

(22) PCT Filed: Apr. 28, 2023
(86) PCT No.: PCT/IB2023/054440

§ 371 Date: Nov. 5, 2024

(30) Foreign Application Priority Data
May 5, 2022 (PL) P.441099

(51) Int. Cl. A61B 3/113; A61B 3/00; A61B 3/14

(52) U.S. Cl. CPC A61B 3/113; A61B 3/0041; A61B 3/0083; A61B 3/0091; A61B 3/14

(57) ABSTRACT

A device for automated measurements of eyeball deviation and/or vertical and horizontal observation angles, comprising optomechanical system cooperating with image recording and display devices has a screen placed in an integrated housing, in front of which two symmetrically embedded optomechanical modules are mounted on the side arms said side arms are movably embedded on parallel, horizontal guideways arranged perpendicularly in relation to the side arms and driven along these guideways with a servo drive and driving elements, wherein every optomechanical module has a camera operating in the invisible light spectrum, optical tube fixing the lens system, vision switching off system, eye pupil illuminator operating in the invisible light spectrum, where the screen is placed perpendicularly to the axis of the lens system and at a distance that allows acute viewing of the screen and covering the possibly largest part of the eye's field of view.

### TECHNICAL FIELD

The object of the present invention a method and a device for automated measurements of eyeball deviation and/or vertical and horizontal viewing angles, consisting of a screen and movable cameras, mirrors and corrective lenses automatically adjusting to the individual eyeball distance, using the technique of alternating and cyclical projection of graphic patterns on the screen and synchronous eye covering, analyzing and recording adjusting eye movements resulting from the movement of these patterns, dynamically determining new positions of the patterns based on the last recorded directions of adjusting movements of the exposed eye and finally calculating e.g. vertical and horizontal strabismus angle based on the last registered graphic positions patterns on the screen after the cessation of adjusting movements and based on relevant information about the patient's individual pupillary distance and visual field distortions caused by the patient's refractive lenses.

### BACKGROUND ART

In ophthalmology and orthoptists' offices, the time-consuming one-sided and alternating “eye covering” test (Eng. cover test) with the use of prismatic strips is commonly used to assess the actual value of the strabismus angle. This examination may be unreliable and subject to error resulting from the subjective, visual assessment of eye movements performed by the examiner, depending on his skills and experience. This examination is time-consuming and requires good cooperation with the patient, and in the case of complex strabismus and/or patients having difficulties in cooperation with the doctor and/or optometrist, it requires the help of additional persons.

A commonly known device for measuring strabismus angles is the optical-mechanical synoptophore. This device in its basic form consists of two rotating optical modules placed close to the patient's eyes and used for projection and observation of static images. The structure of the images allows them to be combined into one image as a result of the appropriate positioning of the optical modules and synoptophore arms by the patient in order to combine them into one image. With the progressive rotation of the optical modules, the images are alternately blanked, forcing adjusting movements of the patient's eyes. The optometrist, based on the visual assessment of the adjustment movements and the current angles of rotation of the optical modules read from the scale or from digital encoders, assesses the angle of strabismus. Synoptophores are devices that are complicated to use and require extensive experience of the examiner-optometrist, and the test result depends on the patient's subjective vision impression.

The solution CN101433456A discloses an intelligent synoptophore used for diagnosing the type and degree of strabismus, equipped with cameras, LCD screens and eye movement detection software to facilitate the work of the optometrist. In the synoptophore, the position of the image is changed mechanically by means of electric motors. Such a solution does not allow for proper calibration of the optical systems of the cameras, i.e. the method of translating the intensity of the eye movement into the angle of the drives position, in this case the stepper motors, which properly position the optical modules.

The CN105942966A discloses a strabismus self-detection system based on a digital synoptophore, improving the autonomy of the device work and its effectiveness in detecting and diagnosing strabismus. Similarly to the solution CN101433456A, the inability to calibrate the optical systems of the cameras leads to inaccurate measurement result. In addition, this device is not used to measure the angle of strabismus, but only to detect strabismus, what results in the lack of accurate diagnostics.

A commonly used method of assessing the strabismus angle is the observation of the position of a point light reflex on the pupillary plane. This method is used by some automated devices. There are also many descriptions in the scientific literature regarding the techniques of measuring the position and movement of the eyeball based on the analysis of the position and shape of the pupil in camera images.

The solution WO2011021936A1 discloses a device and method for automatically determining the strabismus angle by performing a reflection test. The device directs at least one beam of visible light to the patient's eyes and requires the eyes to be focused on it, then using at least one imaging device it analyzes the reflected light to detect the fixating eye, and to perform a reflex test on both eyes to estimate the angle of strabismus. The reflex test consists in applying at least two light sources in known alternating positions and measuring the position of their reflections on the corneas in both eyes. The recorded reflections are used to obtain the coordinates of the center of both corneas and to estimate the coordinates of the center of the pupil, which are further used to estimate the angle of strabismus. For the fixating eye, the angle is calculated between the optical axis of the eye passing through the center of the cornea and the center of the pupil, and the visual axis passing through the center of the cornea and the center of the eye's visual field. This angle is then converted to the strabismus angle.

The RU2669734C1 solution shows a system for calculating the strabismus angle in patients with a relatively low relative error equal to Δα=0.4°. The measurement process described in the solution is non-autonomous and requires a lot of operator involvement. For each patient, it is necessary to carry out a calibration process by applying a measuring line to the patient's face in order to match the number of pixels in the image to the actual distance. For the measurement, it is used a camera and a light source, which the patient looks at, and the patient's head is fixed in the forehead-chin support. An image is recorded on which the distance between the vertical lines passing through the outer and inner corners of the fixating eye is measured, the distance between the center of the pupil and the glare from the light source on the squinting eye, then the radius of curvature of the sclera and the strabismus angle is calculated using the mathematical formulas presented in the solution.

In the state of the art, from publication CN110575132A, there is also known a method of calculating the degree of strabismus based on a photograph of the eye and using artificial intelligence algorithms, i.e. deep learning in neural networks. In this solution, the image of the pupil and the point of reflection of the corneal light in the image of the pupil are analyzed and, based on its position, it is determined whether there is strabismus and, if so, its degree. The ratio of the shifted distance between the corneal light reflection point and the center of the pupil to the radius of the pupil allows the degree of strabismus to be calculated. This solution only discloses a method of measurement, it does not present the construction of executing devices used for recording and image analysis, and the measurement is based on subjective optometry.

Another publication US2014268051A1 describes a method and apparatus for detecting strabismus in an eye image recorded by a camera using the reflection of a light source on the eye. The examined person looks at a target shifted from the light source at a known distance in the range of 5 cm to 10 cm. The shifted target provides the focus of the examined person's gaze, who can be instructed to look at the target while capturing the image (and/or reflexively focus on the target). By knowing the examined person's relative position, light source, and target, reference data can be determined that can be compared with data determined from the captured image to detect strabismus. The reference data represents the expected reflection shift distance in the eye without strabismus. The eye image recording device may be a camera, smartphone, laptop, ophthalmoscope and/or any other device capable of capturing images and having computing capabilities (processor). The solution does not describe the determination of strabismus angles, it does not describe the calibration of the device and the method of obtaining reference data on the basis of which the threshold value defining the border between a healthy person and a person with strabismus is determined.

Observation of the location of the spot light reflex on the pupillary plane can also be used in exercises aimed at reducing the strabismus angle. From the description of CN112807200A there is known a device for the treatment of strabismus, in which the degree of strabismus is gradually reduced by performing visual training. The device has at least one camera and two LED light sources working together in the near-infrared band, polarizing filters for each eye separately and one display. The cameras are used to detect the position of the eye on the image. The near-infrared light source provides illumination that is received by the near-infrared camera and produces corneal reflections as reference points for calculating eye movement. The device does not determine strabismus angles and it is not possible to automatically adjust it to the individual anatomical features of the patient.

Yet another solution, CN112336301A, presents a device for measuring strabismus angles with filters covering the eyes alternately, in which the spatial position of the eye axis is determined by a stereovision system based on the position of the pupils and corneal reflexes. These data are used for initial individual calibration of the relationship between the coordinates of the point on which the eye fixates on the screen and the movement of the pupil on the image on the entire display plane of the fixation targets. Several points at known positions on the screen are used for initial calibration. In the next stage of the one-point calibration, the individual Kappa angle for each patient is determined, which determines the deviation of the geometric axis of the eye from the axis of real vision. Then for any position of the fixation target and calibration model. The device according to the description serves to reveal the type of strabismus and its direction tendencies. The description does not disclose detailed information on the method of determining the angle of strabismus other than the analysis of the rotation of the iris of the eye and/or the calculation of the angle between the viewing axis of the uncovered and covered eye, which would be equivalent to using a prism of appropriate power. The axes should coincide in the absence of strabismus. It has not been indicated whether the method of determining the location of fixation targets during the examination is manual or automatic and on what basis it is performed. The description indicates the possibility of automating the device and obtaining similar results. It is not disclosed whether the device takes into account the significant impact of individual refractive error on the test result and how effectively the patient's image fusion is broken down.

Methods based on the analysis of reflexes on the plan of the pupil, reflexes from the cornea and deeper layers, or based on the shape of the pupil on the images are unreliable, because they do not take into account the real path of light from the observed object to the macula on the retina, or various, often far from ideal, anatomical structure of the eye, even though the Kappa angle has been estimated. Both methods can only be an initial estimative diagnosis showing the presence and type of strabismus.

The device known from the description of EP2403260A2 is the so-called 3D glasses, the principle of which consists in alternately covering the eyes with the use of electronically controlled liquid crystal panels and synchronously displaying a different image on the screen for the left and right eye in order to obtain the impression of spatial vision. Alternatively, polarizing glasses are used, and the image on the screen must also be properly polarized for the left and right eye. Similar glasses are used for fusion exercises in patients diagnosed with strabismus.

Patent description CN104799998A presents an optical device for correcting strabismus based on 3D glasses imaging, where the patient himself changes the image display location by means of buttons so as to experience stereoscopic vision. The device is not used to measure the angle of strabismus and accurate diagnostics.

From solutions US2016143527A1 and U.S. Pat. No. 9,572,488B2, goggles with integrated IR cameras are known, used to observe the movement of the pupils, and LCD screens for alternately covering the eyes. They enable the automated Hess test to be carried out, i.e. only the assessment of eyeball mobility. The described device and method of measurement provide an objective and repeatable measurement of eye movement.

VR (Eng. Virtual Reality) goggles, with an integrated eye-tracking system (Eng. eye-tracking), are widely available. These solutions are used to control virtual interfaces or in virtual and augmented reality techniques for visualization, multimedia or entertainment purposes.

The solution according to the description of CN112107416A presents a device for visual imaging with strabismus correction, based on VR goggles. A module for acquiring video information is mounted to the front part of the body, and two image display modules and a module for image data processing are mounted inside the goggles. Real-time video information about the external environment can be processed in such a way that the vision of a strabismus patient is not disturbed and he can comfortably assimilate the environment. Image transformations are based on recorded eye movements and the results of separate medical examinations. The solution does not disclose the details of system calibration and the method of transforming images to the patient's strabismus angles and is not a diagnostic device.

On the other hand, the descriptions of CN111820860A and WO2020184775A1 present similar solutions for measuring the angle of strabismus, based on the display of stereoscopic images, e.g. in VR goggles with a system for recording the position and/or movement of the eyes. The computer controls the position of the indicators displayed on the screens so that they can be visually followed while the left and right images of the VR glasses are alternately closed and/or blocked. Cameras are used to take pictures of the pupils while closing one of the images, and the computer is used to assess the movement of both pupils simultaneously and determine a new pointer shift based on the analysis of the movement of the squint eye. The pointer moves are made until the pupil movement stops and the last position of the pointer is used to determine the strabismus angle. Publications indicate that the devices are not dependent on the subjectivity of the examined person, and the measurement results are accurate. Both solutions, however, omit the aspect of field of view calibration in relation to individual anatomical features, in particular the distance between the pupils and the distance of the eye from the screen, which in the case of small changes and small distance of the eyes from the screens can significantly affect the measurement result. The publications also do not disclose whether the possible calibration will allow to take into account the patient's refractive errors and distortions in the patient's field of view introduced by the lenses, which have a significant impact on the degree of convergence and the final results of angle measurements.

From publication CN109288493A, a device and a method for diagnosing strabismus using graphic patterns on screens, an infrared and/or visible light camera, a mechanically movable eye cover and a head support with installed prism bars are known. The device uses two independent screens to display the image and perform the examination for far distance of 6 meters and for near distance of 0.33 m. The solution does not reveal the degree of automation of this measurement. According to the description, the device performs an initial strabismus presence test and evaluates its direction, and then a prism with an estimated degree obtained in the qualitative experiment is placed on the prism frame just in front of the patient's eye. The device automatically covers the eyes and records the movement of the center of the pupil in the images. The device omits the issue of measuring the strabismus angle and techniques for calibrating the position and field of view.

From the description of WO2017123086A1, a method and a computer system for determining the angle of strabismus are also known. The method consists of placing the patient in front of an eye tracking device and in front of any image display device at varying distances from 0.3 to 5 meters for near and far distance vision tests, displaying a small graphic element on the screen in one and/or nine main viewing directions, measuring by a computer the line of sight of the person's eyes with an eye tracking device and calculating the strabismus angles between the eyes by calculating the difference in the horizontal and vertical line of sight directions. The method of selecting the line of sight directions is not specified in detail. In the device, it is possible to use infrared filters to observe hidden strabismus and two infrared cameras with illuminators, each of which observes both eyes in real time. This publication also indicates the possibility of using corneal reflections and an eye model to assess the shape of the eye and the position of the eye axis in space. The advantage of this solution is the observation of eye movement and head movement independently, which facilitates examinations in pediatric patients. This solution also omits the technique of calibrating the device without the patient's participation and ensuring the possibility of adjusting the measurement to the distance between the pupils and correcting the refractive error, what is important during the examination.

Yet another solution, known from the patent description KR101825830B1, presents a system and a method for measuring the angle of strabismus, using the covering test and eye movement analysis during the observation of graphic patterns displayed on a screen (e.g. mobile phone, tablet, LCD screen). The test can be performed for long and short fixation distances (30 cm-1 m), also in persons with visual impairment, but the solution does not describe the impact of this defect on the method and effects of the measurement. The cover is moved manually by the patient, and/or it can be glasses with blinds put on the head, which can additionally have a module for recognizing the position and distance between the pupils. The determined pupillary distance is related to the positions of the patterns displayed on the screen. The device calculates the strabismus angle on the basis of successive photographs of both eyes and recognizing the moment of cessation of adjusting movements. During the examination, the eye follows the templates moving on the screen, but the algorithm for planning their positions (points P2, P3) and whether it is an automatic or manual process is not described. The device shown is not an integrated construction and there are no known calibration procedures for any viewing direction and any visual impairment. The determined angle of deviation of the eyeball (theta angle) depends on the position of the template on the screen, at which the adjusting movement of the eyes does not occur, but the solution does not take into account other significant factors affecting the number of prismatic diopters of the calculated prism, such as corrective lenses.

From the description of US2015265146A1, a device for diagnosing and quantifying the degree of strabismus is also known. The device includes a beam, a video camera, a light source to generate Purkinje reflections, and a computer. The patient stares at targets at known angles (e.g.−30°, 0°, +30°) while the video camera records the patient's eyes. The image is sent to a computer that analyzes each image frame. Pupils and Purkinje reflexes are identified. The described method is effective but requires the examiner to be a well-trained and experienced operator. The patient's head is not fixed in position relative to the video camera, and the patient holds the head still without the use of any external device. It follows that the described device is not suitable and/or is difficult to use in the case of measuring strabismus in children. The device can be used both for strabismus screening and as a quantitative tool for surgery planning, reducing the number of surgeries needed. The strabismus angle is calculated in this method by means of linear regression using the Hirschberg ratio, which means that the described method is not accurate.

From the solution CN107898429A, there is known a solution that allows to quickly carry out screening test whether there is a strabismus along with its identification—recessive strabismus and/or manifest strabismus, horizontal strabismus and/or vertical strabismus. The examined person looks at the optotype placed at a distance of 33 cm and/or 5 m, and the image is recorded by a thermal imaging camera. It is a type of test based on the principle of covering an eye. Video recordings recorded while covering the eye are transferred to the computer. In order to obtain the appropriate result of the screening test, the table included in the patent description is used. The solution does not allow for accurate diagnostics and does not reveal the possibilities and principles of optical systems calibration.

### DISCLOSURE OF THE INVENTION

In accordance with the knowledge of the inventors, there are no methods or devices known in the state of the art that enable accurate, fully automatic measurement of eye deviation and/or vertical and horizontal viewing angles, taking into account, during the measurement, significant corrections for individual pupillary spacing and distortions introduced by additional lenses adapted to the patient's individual refractive error, operating based on the patient's observation of the patterns displayed on the screen during alternate cyclical switching off the vision of one and/or the other eye, and the positions of these patterns are dynamically determined based on the analysis of current pupil deviations from the fixation position recorded on cameras' images.

The term “patient” includes both persons diagnosed for medical purposes and as well all persons who need to measure their individual characteristics of the vision system in order to best match them with stereoscopic imaging devices, e.g. interfaces to virtual and augmented reality, and to ensure better comfort of use or work on these devices.

The terms “switching on the vision” and “switching off the vision” should be understood as the use of any technical means that result in partial and/or complete blocking of light access to the eye, as a result of which there are no objects for fixation in the eye's field of view. These terms do not apply to the suppression of vision resulting, for example, from long-term diseases and the dominance of one eye over the other.

The aim of the invention is to propose a solution based on the real course of light rays to the eye spot in the axis of vision and will eliminate the need to estimate the position of this axis relative to the geometric axis of the eye, as is the case in many of the above-mentioned solutions.

On a daily basis, a person with strabismus, without the use of appropriate prismatic correction, switches the sense of vision between the eyes and/or chooses one dominant eye, which, when trying to focus on an indicated object, causes alternating adjusting eyeballs movements. After applying the traditional prism correction, alternating observation of the indicated object should not cause the patient's adjusting eye movements. Similarly, the adjustment movements disappear in the synoptophore examination as a result of the appropriate positioning of the optomechanical modules and the images displayed in them, and/or by selecting the appropriate prism during the examination using a traditional prism bar.

A mechanical synoptophore and/or prism bars can be replaced by an image dynamically moved on the screen surface based on the analysis of eye adjustment movements and synchronously switched off and/or covered alternately for each eye separately, as a result of which the patient regains the impression of spatial binocular vision, and the adjustment movements cease. Switching off the vision can be achieved by using mechanical and/or electronic covers or by using separated areas of vision on the screen with the possibility of their switching off. In addition, if the image movement is related to the lens correction of the patient's own refractive error and is related to the patient's individual eye spacing, it is possible to accurately determine the angle between the axes of both eyeballs.

Therefore, the solution according to the invention imitates to a large extent the classic cover test with use of prism bars, however it can be fully automated and does not have the disadvantages of this and other previously presented methods, and also is not based on unreliable methods based on the analysis of light reflections in the structures of the anterior segment of the eye, or based on the analysis of the shape of the eye structures.

A device for automated measurements of eyeball deviation and/or vertical and horizontal viewing angles, comprising optomechanical system cooperating with image recording and display devices is characterized in that it has a screen placed in an integrated housing. In front of the screen two symmetrically embedded optomechanical modules mounted on the side arms are placed—constituting an optomechanical system. These modules are mounted on the side arms. And the side arms are movably embedded on parallel, horizontal guideways arranged perpendicularly in relation to the side arms and driven along these guideways with a servo drive and driving elements—e.g. a toothed belt or a lead screw. Every optomechanical module has a camera operating in the invisible light spectrum e.g. near-infrared, a system for switching off the vision, e.g. a mechanical cover, an optical tube fixing the lens system, and an eye pupil illuminator operating in the light spectrum invisible to the human eye. The screen is placed perpendicularly to the axis of the lens system and at a distance that allows acute viewing of the screen and covering the possibly largest part of the eye's field of view. Moreover a selective element is placed between the optical tubes and the screen e.g. glass coated with selective filter or beam-splitter enabling the reflection of both pupil images in the invisible light spectrum towards the cameras, but at the same time enabling continuous visual observation of the screen. The illuminators are mounted in such a way as to illuminate the entire pupil of the eye and at the same time not to cause light reflections on the lens surfaces visible in the cameras images. The important thing is that the screen, camera, vision switching off system, servo drive and drive elements are connected and controlled by a computer.

The exceptionally preferably integrated housing has a permanently mounted head stabilizer, e.g. in the form of a stabilizing frame and/or the appropriate shape of the housing, enabling tight seating of the face of the examined person, at the same time minimizing the amount of diffused external light falling on the retina.

The possibility of changing the parameters of the lens system is also advantageous, e.g. by changing the focal length of the lens system, axial movement of the lenses, screen movement and/or the movement of both of these elements in relation to the eye, and/or installing additional trial lenses in order to correct the patient's refractive errors that have a significant impact on the correct assessment of the position of the optotypes, and thus on the result of measurements of deviation of the eyeballs and/or vertical and horizontal viewing angles, including strabismus angles.

In another preferred variant, each of the side arms on which the optomechanical modules are mounted additionally has at least one vertical guideway, perpendicular to the horizontal guideways, embedded in an integrated housing. The horizontal and vertical guideways together enable the module to move horizontally and vertically independently for the left and right eye.

In another preferred variants, each optomechanical module may have its own integrated screen, which allows greater control over the cyclic display of the optotypes and allows complete separation of the field of view of the left and right eye, and eliminates the need for additional covers.

The essence of the invention is also a method of automated measurements of angles of automatic strabismus using the device. The method uses the measurement of the observer's pupillary distance and a model of geometric distortions of the field of view. In this method, firstly, the size of vertical and horizontal distortions resulting from the application of lens systems in front of the observer's eye, including additional correcting lenses, i.e. spherical and/or cylindrical, is determined, enabling the observation of the screen at an infinite distance. The lens system is then adapted to the individual refractive error of the patient, e.g. by placing in front of his eyes trial lenses adapted to his individual refractive error. Next, through the movements of the servo drives and using the system of alternated switching off the vision, the main axes of the optomechanical modules are centered in relation to the position of the pupils of the left and right eye, thus obtaining information about the observer's actual horizontal pupillary distance and determining on the screen the position of the fixation points located in front of each eye, preferably at a mutual distance on the screen corresponding to the distance between the pupils. Then, during the alternating cyclic switching off of the vision of one of the eyes, an image pattern is displayed on the screen surface in a fixed position straight ahead for one eye and a pattern in a variable position relative to the point of fixation straight ahead for the other eye, with the variable position determined in each cycle based on the intensity and the direction of the adjusting movement of the pupil recorded by the camera at the moment of switching on its vision. After the pupil movements cease and the graphic patterns are set in the patient's natural axes of vision of both eyes, the last changing position of the pattern is corrected by the values of vertical and horizontal distortions introduced by the lens system, using the model of geometric distortions of the field of view. Finally, based on the knowledge of the corrected position and distance from the screen, the vertical and horizontal viewing angle of the graphic pattern is determined.

Exceptionally preferably the parameters of the lens system are changed e.g. the trial lenses are selected to force the screen to be seen at near distance in accordance with the actual distance from the screen, and the fixation points for the left and right eye are moved towards the axis of symmetry of the device so as to force seeing the screen at a near distance.

A variant is also advantageous when the variable position of the pattern is determined in each cycle on the basis of the intensity and direction of the adjusting movement of the pupil of the examined eye, registered by the camera at the moment of switching off its vision, exactly at the moment when the other eye begins to fixate straight ahead on a immobile graphic pattern.

Preferably, it is envisaged that the graphic patterns represent the same three- dimensional object, the visualization of which on the screen takes into account geometric transformations separately for the left and right eye, and causes the effect of stereoscopic observation of a real three-dimensional solid.

Preferably, it is also provided that immediately before and during the measurement, an additional image is displayed on the screen in the background, e.g. a mountain landscape and/or a starry sky, enhancing the observer's impression of spatiality, facilitating the switching off of binocular accommodation and looking to an infinite distance.

Preferably, the optical tube with the lens system and/or the screen are mounted on additional guideways and are driven by computer-controlled servo drives, the movement of which is carried out perpendicularly to the screen and allows changing the focus point of the image and adjusting it to the refractive error of the eye.

Preferably, the cameras have an additional optical module and a structured light projection module, enabling projection on the retina of the eye and observation of the image of this light by the cameras and automatic measurement of the refractive error. The measurement result, in turn, enables the automatic adjustment of the optomechanical module to the refractive error of the eye before testing the angles of deviation of the eyeballs and/or the vertical and horizontal viewing angles. The device according to the invention allows, for example, as in traditional examinations, to alternately cover the eyes and perform cyclical tests for one eye fixating on a immobile graphic object placed in front of the eye, while the graphic object for the other eye moves proportionally to the intensity of the pupil adjustment movements in the image recorded by the appropriate camera at the time of switching on the vision.

The design of the device according to the invention enables the patient to observe the screen and, at the same time, continuously record the images of both pupils by the camera system. Each successive movement of the graphic pattern is automatically calculated on the basis of two essential pieces of information: individual pupillary distance in the vertical and horizontal direction and the intensity of the last recorded adjusting movements of the examined eye in the vertical and horizontal direction at the moment of switching on its vision. The device does not perform a repetitive fixed cycle of operations, but dynamically selects pattern positions based on the intensity of the patient's eyes movements. Cooperation with the patient comes down only to his ability to track and focus his eyes on the graphic pattern displayed in various positions on the screen. The moment of cessation of adjusting movements is equivalent to stabilizing the position of graphic patterns and for well-cooperating patients it may end after a dozen or so seconds. The final position of the optotypes in conjunction with the model of visual field distortions and data on the individual refractive error and pupillary distance make it possible to determine deviation angles of the patient's strabismus.

The device according to the invention, as in the case of traditional methods, enables the examination of strabismus angles for far and near distance, by appropriate adjustment of the parameters of the lens system and/or appropriate settings of the screen position. Similarly, at any time during the examination on the device, the vision one of the patient's eyes is always switched off, which causes the image fusion break-up effect, which is crucial for the correct measurement.

When used for typical medical diagnostics, the undoubted advantage of the invention is the possibility to carry out measurements repeatedly in stable and repeatable conditions, and in a constant position of the patient's head during the examination, which is especially important in poorly cooperating and pediatric patients. Additionally, the examination can be performed much faster and without the involvement of additional medical personnel (holding the child's head, holding an additional prism bar when calculating vertical or diagonal deviations coexisting with horizontal). The solution according to the invention enables to partially or completely make the result independent of the researcher's experience and participation during the measurement. The patient will only be asked to focus on the selected patterns. The use of the device according to the invention may also translate into better and more accurate results of strabological surgical procedures, because the measurements of the angle of strabismus are the basis for making the correct diagnosis of the type of strabismus, the degree of muscle deviation, the extent of this deviation, but also on the basis of these measurements the scope of the adopted surgery technique on the extraocular muscles is planned.

When used for non-medical diagnostics, the undoubted advantage of the invention is the possibility of using it to personify the settings of devices used to create virtual or augmented reality, e.g. with the use of appropriately designed goggles. Such devices can then be used to create, for example, virtual workspaces, virtual operator's panels to control machines, or 3D graphics presentations for entertainment purposes. Personification refers to adjusting to the individual anatomical features of the visual organ and can translate into long-term and comfortable use of these devices without adverse side effects, such as fatigue, headache or dizziness.

### EXAMPLES

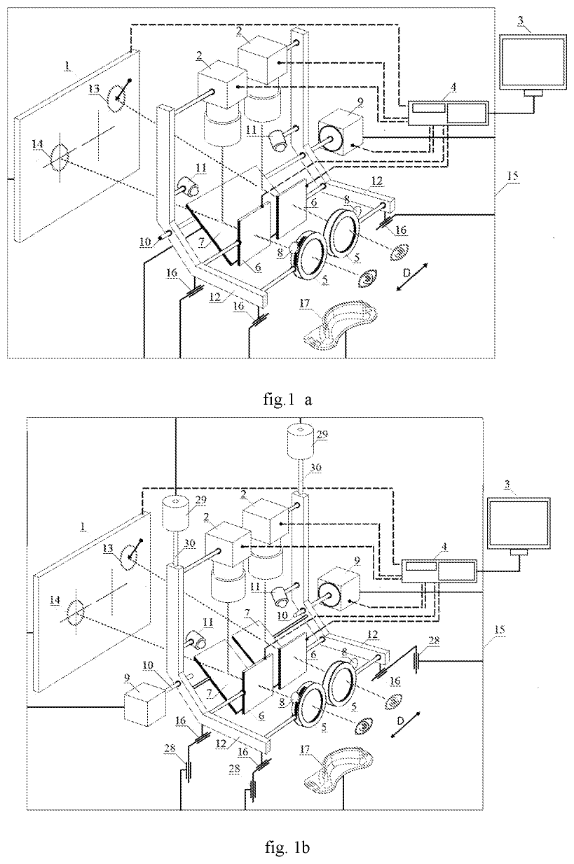

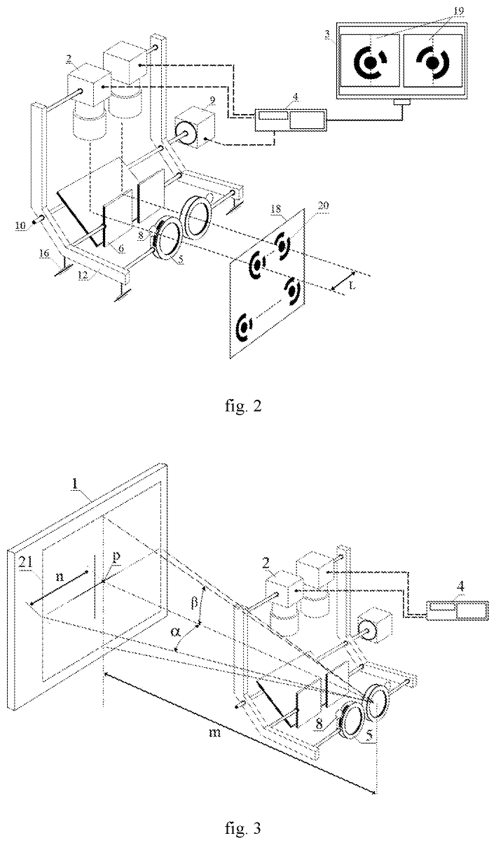

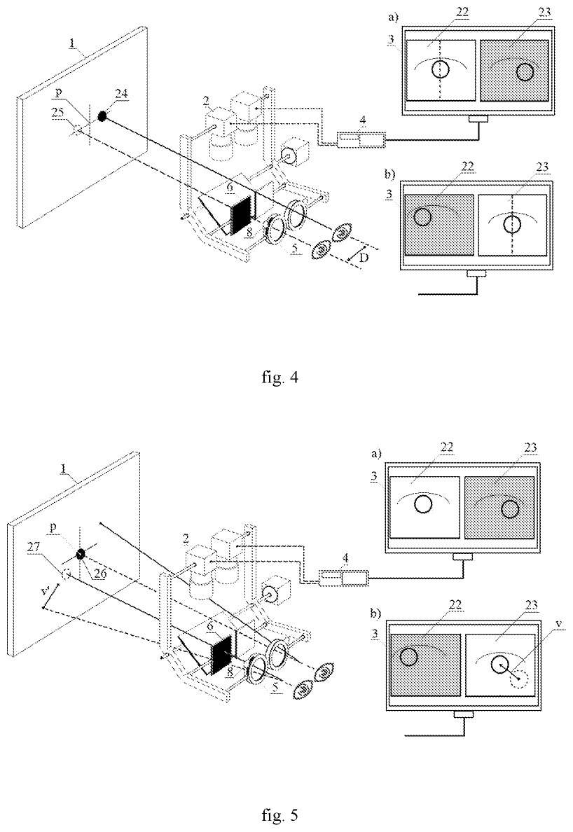

The solution according to the invention is disclosed in detail in the examples of implementation and application as well as in the drawings, where FIG. 1a and 1b show schematic diagrams of the operation of the device and its construction in two variants-basic and equipped with vertical guideways, FIG. 2 shows the calibration of the optical axes spacing, FIG. 3 shows the concept for calibrating the field of view. FIG. 4 shows schematically the measurement of pupillary distance a) with the left eye vision switched off and b) with the right eye vision switched off, and FIG. 5 shows the strabismus angle measurement cycle for the near distance of the right fixating eye a) with the right eye vision switched off and b) after switching on the vision of the right eye.

### Example 1

According to the diagram shown in FIG. 1, the device for measuring the deviation of the eyeballs and/or the vertical and horizontal viewing angles consists of integrated in one housing 15: a screen 1 in any technology for displaying image patterns, a computing unit 4, e.g. a PC computer, a operator panel 3, permanently attached to the head stabilizer housing 17, as well as two symmetrically made and embedded optomechanical modules. Each optomechanical module consists of sub-assemblies fixed to element 12: a camera 2 operating in the near-infrared band and connected to the computer 4 for image acquisition and analysis, vision switching off system 6 (blackout of part of the screen 6a or a cover 6b) controlled by the computer 4, an optical tube 5 mounting the replaceable trial lenses 8, the illuminator 11, e.g. a LED operating in the near-infrared band, and the mounting element 12 movably mounted on the guideways 16 so as to enable precise movement of the entire module along these guideways. In a variant of the device (shown in FIG. 1b) it is also provided that each of the side arms 12 on which the optomechanical modules are mounted may be equipped with at least one vertical guideway 28 perpendicular to the horizontal guideways 16, embedded in the integrated housing 15. Horizontal guideways 16 and vertical guideways 28 together enable the module to perform movements in the horizontal and vertical directions independently for the left and right eye. Both mounting elements 12 move independently in relation to each other by servo drives 9 for horizontal axes and servo drives 29 for vertical axes, controlled by the computer 4 and driving elements 10 and 30, respectively, e.g. lead screws, together allowing precise determination of the distance between the main optical axes of the cameras 2 and the trial lenses 8. The illuminators 11 are permanently mounted in such a way as to illuminate the entire pupil of the eye and at the same time not to cause reflections of light on the surfaces of the lenses 8, which hinder the observation of the pupil by the cameras 2. The construction of the optomechanical modules should allow movement in such a range that the distance between the axes of the lenses 8 can be adjusted to the typical distance D between human pupils. The screen 1 is placed perpendicularly to the axis of the trial lenses 8 and at such a distance from the optical tubes 5 as to cover as much of the eye's field of view as possible. Between the lenses 8 and the screen 1, a selective mirror 7 is placed, enabling the reflection of both pupil images in the infrared band towards the cameras 2, but at the same time enabling continuous visual observation of the screen 1. In an alternative variant (as in FIG. 1b), one selective mirror 7 can be separated into two, symmetrically attached to each optomechanical module separately. In contrast to the variant according to FIG. 1a, where the vision is switched off by blackout of the part 6a of the screen 1, in the alternative variant (as in FIG. 1b), in order to switch off the vision of a given eye, it is planned to use covers 6b, e.g. LCD, separately for the right and left eye, attached to the arm 12. The covers are attached in such a way that no part of the screen 1 is visible in the field of view of the covered eye.

In the computer 4, software is implemented that controls the operation of the servo drive 9, software for analyzing the intensity and direction of eye adjusting movements, controlling the display of a moving image pattern 13 for the examined eye and an immobile image pattern 14 for the other eye, as well as software that controls the blackout of the part 6a of the screen 1 (as in FIG. 1a) and/or the operation of covers 6b (as in FIG. 1b). Graphical patterns 13 and 14 represent the same object of small size and shape facilitating visual fixation, and it is a flat graphic, e.g. vector or raster. As an alternative, the graphic patterns 13 and 14 represent the same three-dimensional object whose geometrical transformations separately with respect to the left and right eye cause the effect of stereoscopic observation of a real three-dimensional solid.

The calibration of the device is two-stage, including the calibration of the spacing of the optomechanical modules and the calibration of the field of view in relation to the viewing angles. The first stage, in accordance with FIG. 2, consists in finding the relationship between the actual spacing L of the optical axes of the optical tubes 5 and the movement of the servo drive 9 and the driving elements 10. For this purpose, it is registered an image of the distance standard 18, made of at least two pairs of flat markers 20 placed at distances corresponding to typical minimum and maximum pupillary distances, with the left camera 2 only observing the left marker and the right camera 2—the right marker. For each pair of markers 20, the spacing of cameras 2 is set so that on images 19 from cameras 2 the markers are exactly in the middle of the width of these images and the position of the servo drive 9 is recorded. The recorded positions of the drive 9 for all reference distances 20 enable the creation of a linear model of this relation whose parameters are stored on the computer hard drive 4.

The second stage of calibration, in accordance with FIG. 3, consists in determining the mathematical dependence of the position of the farthest points possible to be viewed on the screen from the real angle of their viewing, thus determining the active measurement area 21 and at the same time the field of view. For this purpose, mathematically apparent for the eye changes in the angular size of the measurement area 21, are determined after using the trial lens 5 with an optical power in the range of −10 diopters to +10 diopters, and then from trigonometry the limit angles α and β are determined for the extreme points of the field of view distant from the point p by bx and by, respectively, taking into account the known distance m of the screen 1 from the place where the trial lens 8 is mounted. The dependence of the angles of viewing of the extreme points of the measurement area on the screen 1 from the dioptric power of the installed trial lens 8 creates a mathematical model that makes it possible to correct the determined viewing angles for any points of the measurement area other than the extremes, apparently increased or decreased as a result of the operation of the trial lenses 8. The parameters of the model are saved on the computer hard drive 4. A calibrated system can be used to measure the strabismus angles.

### Description of the Examination:

When starting the examination, the patient rests his head on a properly profiled stabilizing frame 17, the position of which should be adjusted so that the left and right pupils are in the field of view of the left and right camera 2, respectively, and the head remains motionless.

In the first stage of the measurement, the pupillary distance is measured. According to FIG. 4, the vision of the left eye is switched off by obscuring of the left cover 6b, and in front of the right exposed eye at a distance of +33 mm from the center p of the screen 1, a graphic pattern 24 is displayed on which the patient's vision is focused. According to FIG. 4a), the image 22 of the right pupil is registered by the camera 2, and the software in the computer 4 determines its position in relation to the center of this image and, on this basis, determines the direction of movement of the driving elements 10. The right optomechanical module is automatically set together with the mounting element 12 with cameras 2 and trial lenses 8 so that the pupil is in the middle of the width of the image 22. The position of the servo drive 9 is saved on the computer hard drive 4. Similarly, after switching on the vision of the left eye and switching off the vision of the right eye, a dot pattern 25 is displayed in front of the left eye at a distance of −33 mm from the center p of the screen 1. Then, according to FIG. 4b), the image 23 of the left pupil is registered, then the software performs the centering of the pupil on this image by moving the element 12 and the second position of the servo drive 9 is recorded. Based on both recorded positions of the servo drive 9 and on the basis of recorded data from the device calibration procedure, the patient's pupillary distance D is determined and stored on the computer hard drive 4, what is necessary for further examination.

In the second stage of the measurement, according to FIG. 5, the angles of strabismus to near distance are measured for the left eye, while the right eye is always fixated on the graphical pattern located in the center p of the screen 1. Before the examination, trial lenses 8 are placed in the optical tubes 5 of optical power selected individually for the patient so that, as a result of the fixation of the pattern on the screen 1, to obtain the effect of natural vision up to the near of typically 300 mm. Then, a cycle begins in which vision of the left eye is switched off by the cover 6b in order to break the fusion of the patient's images, and a graphic pattern 26 is displayed in the center p of the screen 1 on which the patient's vision is focused. The camera 2 registers the position of the pupil of the left eye that is switched off on image 23 and saves this position on the computer hard drive 4. Then the cover 6b of the left eye is opened and at the same time the cover 6b of the right eye is closed and again the patient focuses his eyes on the dot pattern 26 displayed in the middle p of the screen 1. The pupil position of the left eye after switching on its vision in image 23 is re-recorded, and then the software calculates a vector v for the difference of this position and the position recorded before switching on the left eye. On the basis of this difference, the vector v′ is determined, which is a proportional rescaling of the vector v and denoting the value of movement of the dot pattern 26 on the plane of the screen 1 to the new position 27, and thus the cycle ends. In the new cycle, when left eye vision is switched on, the dot pattern is displayed at the new position 27 determined in the previous cycle. Similarly, a shift vector v is determined in the pupil image 23 of the left eye when its vision is switched on, which is used to calculate the position of the pattern in the next cycle. The proportion between the vectors v and v′ is determined experimentally in such a way that in subsequent cycles the adjusting movement of the switched-on eye does not increase. The cycle is repeated until the pupil shift vector v of the switched-on left eye reaches the set minimum value expressed, for example, in image pixels, and thus the moment of cessation of the adjusting movement of the left eye is detected by the software. The last position of the dot pattern 27 determined and saved in the computer 4 is corrected on the basis of a known, calibrated model of the dependence of the viewing angle and the dioptric power of the trial used and is converted into actual angles of strabismus, vertical β and horizontal α. The course of measurements with the preview of pupil movement and the measurement results are visualized on the operator's panel 3.

### Example 2

The solution is as in the example 1, but on a calibrated device, after the pupillary distance D measurement, trial lenses 8 with optical power selected individually for the patient are placed in optical tubes 5, so that as a result of fixating the pattern on the screen 1, the effect of natural vision to infinity is obtained. Then, the angles of strabismus to distance are measured, while the fixed position of the graphical pattern 26 displayed on the screen 1 for the right eye is always shifted to the right in relation to the center p of the screen 1 by half of the measured distance D between the patient's pupils.

### Example 3

A solution as in the example 2, but the measurement of strabismus angles is made for the right eye, while the left eye always fixates on the stationary dot pattern 26 shifted on the screen 1 in relation to its center p to the left by half the distance D between the pupils.

In the optimal variant—for each examination—it is envisaged that immediately before and during the measurement, an image and/or background is also displayed on screen 1, enhancing the observer's impression of spatiality and facilitating relaxation of accommodation.

### Example 4

The solution as in the example 1, but the centering of the lens systems is carried out simultaneously for the horizontal and vertical directions in accordance with the applied system of guideways 16 and 28 according to FIG. 1b, and the pupillary distance is calculated taking into account additionally the difference in the position of the pair of drives 9 and the pair of drives 29.

### Example 5

The solution as in the example 1, but instead of a single immobile screen 1, two independent screens 1b are used for the left and right eye, permanently connected to the left and right arm 12, respectively. Such a solution for near and far distance measurements enables to determine the position of the graphic standards 13 and 14 always in relation to the center of the screen 1b, which is calibrated once against the axis of the optical tube 5. The solution is shown in FIG. 6. The advantage of the solution is also the possibility of integrating each optomechanical module with display 1b in one hermetic housing.

### Example 6

The solution as in example 5, but in each optomechanical module, instead of a replaceable trial lens 8, a movable optical tube 5 with a lens system 8b and/or a movable screen 1b is used, mounted on additional guideways 31 and/or 32 and driven by servo drives 33 and/or 34 controlled by a computer 4, the movement of which is perpendicular to the screen 1b and allows for the change of the point of the image sharpness and adjusting it to the refractive error of the eye. One optomechanical system according to the solution is shown in FIG. 6.

### Example 7

The solution as in example 6, but the cameras may have an additional optical module 35 and a structured light projection module 36, enabling the projection on the retina and observation by the cameras 2 of the image of this light and automatic measurement of the refractive error. The measurement result, in turn, enables automatic adjustment of the optomechanical module to the refractive error of the eye before examining the strabismus angles. The solution is shown in FIG. 7.

1. A device for automated measurements of eyeball deviation and/or vertical and horizontal observation angles, comprising optomechanical system cooperating with image recording and display devices, characterized in that it has a screen (1) placed in an integrated housing (15), in front of which two symmetrically embedded optomechanical modules are mounted on the side arms (12) said side arms (12) are movably embedded on parallel, horizontal guideways (16) arranged perpendicularly in relation to the side arms (12) and driven along these guideways with a servo drive (9) and driving elements (10), wherein every optomechanical module has a camera (2) operating in the invisible light spectrum, optical tube (5) fixing the lens system (8), vision switching off system (6), eye pupil illuminator (11) operating in the invisible light spectrum, wherein the screen (1) is placed perpendicularly to the axis of the lens system (8) and at a distance that allows acute viewing of the screen and covering the possibly largest part of the eye's field of view, moreover a selective element (7) is placed between the lens system (8) and the screen (1), enabling the reflection of both pupil images in the invisible spectrum field of view towards the cameras (2), but at the same time enabling continuous visual observation of the screen, the illuminators (11) are permanently mounted on the side arms (12) in such a way as to illuminate the entire pupil of the eye and at the same time not to cause light reflections on the lens surfaces (8) visible in the images from the cameras (2), moreover the screen (1), the camera (2), the vision switching off system (6), the servo drive (9), the driving elements (10) are connected and controlled by a computer (4).

2. The device according to claim 1 characterized in that the integrated housing (15) has a permanently mounted head stabilizer (17) and/or has a profiled shape that allows tight seating of the examined person's face.

3. The device according to claim 1, characterized in that each of the optomechanical modules has the ability to change the parameters of the lens system and/or a handle for additional trial lenses.

4. The device according to claim 1, characterized in that each of the side arms (12) on which the optomechanical modules are mounted additionally has at least one vertical guideway (28) perpendicular to the horizontal guideways (16) embedded in an integrated housing, moreover it preferably has two symmetrically mounted selective mirrors (7).

5. The device according to claim 1, characterized in that each optomechanical module has its own integrated screen (1).

6. The device according to claim 1, characterized in that the optical tube (5) with the lens system (8) and/or the screen (1), are mounted on additional guideways (31) and/or (32) and are driven by servo drives (33) and/or (34) controlled from the computer (4).

7. The device according to claim 1, characterized in that the cameras (2) have an additional optical module (35) and a structured light projection module (36), enabling projection on the retina and observation by the cameras (2) of the image of this light and automated measurement of the refractive error.

8. A method for automated measurements of the deviation of the eyeballs and/or the vertical and horizontal viewing angles using the device described in claim 1, using the measurement of the observer's interpupillary distance and a model of geometric distortions of the field of view, characterized in that, at first in each the point of the actual field of view, the sizes of vertical and horizontal distortions resulting from the use of lens systems in front of the observer's eye, including additional correcting lenses, spherical or cylindrical, enabling the observation of the screen (1) at an infinite distance are determined, afterwards the lens system is adjusted to the individual refractive error of the observer, then, through the movements of the servo drive (9) and using the alternating vision switching off system (6), the main axes of the optomechanical modules are centered in relation to the position of the pupils of the left and right eye, thus obtaining information about the observer's actual horizontal pupillary distance and determining on the screen (1) the position of fixation points placed in front of each eye, and then, during alternate cyclic switching off of the vision of one of the eyes with the system (6), an image pattern (14) is displayed on the surface of the screen (1) in a fixed position straight in front of one eye and an image pattern (13) in a variable position in relation to the fixation point straight in front of the other eye, wherein the variable position is determined in each cycle on the basis of the intensity and direction of the adjusting movement of the pupil v registered by the camera at the moment of switching on its vision with the system (6), and the last variable position of the pattern (13) obtained after the cessation of pupil movements, is corrected by the values of vertical and horizontal distortions introduced by the lens system using the model of geometric distortions of the field of vision, finally, based on the knowledge of the corrected position (13) and distance from the screen (1), the vertical and horizontal viewing angle of the image pattern is determined.

9. The method according to claim 8, characterized in that the parameters of the lens system are changed and the fixation points for the left and right eye are shifted towards the axis of symmetry of the device so as to force seeing the screen at a near distance.

10. The method according to claim 8, characterized in that the variable position of the image pattern (13) is determined in each cycle on the basis of the intensity and direction of the pupil adjustment movement registered by the camera at the moment of switching off its vision with the system (6), exactly at the moment when the other eye begins to fixate straight ahead on a immobile image pattern.

11. The method according to claim 8, characterized in that the graphic patterns (13) and (14) represent the same three-dimensional object, the visualization of which on the screen takes into account the geometric transformations separately with respect to the left and right eye, causing the effect of stereoscopic observation of a real three-dimensional solid.

12. The method according to claim 8, characterized in that immediately before and during the measurement, an image and/or background enhancing the viewer's impression of spatiality is also displayed on the screen (1).
